(19) United States
(12) Patent Application Publication
(10) Pub. No.: US 2010/0317613 A1
(43) Pub. Date: Dec. 16, 2010

(54) ACTIVITY ENHANCER FOR DETOXIFYING ENZYME

(75) Inventors: Hiromu Ohnogi, Shiga (JP); Yoko Kudo, Shiga (JP); Hiroko Nakahara, Shiga (JP); Tatsuji Enoki, Shiga (JP); Ikunoshin Kato, Shiga (JP)

Correspondence Address: BROWDY AND NEIMARK, P.L.L.C.;624 NINTH STREET, NW, SUITE 300, WASHINGTON, DC, 20001-5303, US

(73) Assignee: TAKARA BIO INC., Otsu-shi (JP)

(21) Appl. No.: 12/445,784

(22) PCT Filed: Oct. 11, 2007
(86) PCT No.: PCT/JP2007/069820

§ 371 Date: Apr. 16, 2009

(30) Foreign Application Priority Data
Oct. 16, 2006 (JP) 2006-281731
Jan. 12, 2007 (JP) 2007-004610

(51) Int. Cl. A61K 31/715; A61P 25/00

(52) U.S. Cl. 514/54

(57) ABSTRACT

It is intended to provide a drug, a food or a feed which has an effect of enhancing the activity of a second-phase detoxifying enzyme and an effect of increasing intracellular glutathione content.

### TECHNICAL FIELD

The present invention relates to a drug, a food or a feed which has an effect of enhancing a phase II detoxifying enzyme activity and an intracellular glutathione content.

### BACKGROUND ART

Foods, water, atmosphere and chemical drugs that we ingest every day contain components unfavorable for the living body. Such components are recognized as foreign objects in the living body, and then, mainly metabolized in the liver. Xenobiotic metabolism in the liver consists of phase I and phase II. In phase I, xenobiotic substances undergo oxidation, reduction or hydrolysis by the action of a so-called phase I detoxifying enzyme, such as cytochrome P450 or monooxygenase. In phase II, the xenobiotic substances metabolized in phase I and other xenobiotic substances are conjugated or reduced by the action of a so-called phase II detoxifying enzyme, such as glutathione S-transferase (hereinafter sometimes, referred to as “GST”), quinone reductase (hereinafter sometimes, referred to as “QR”), UDP-glucuronosyltransferase (hereinafter sometimes, referred to as “UGT”), glutathione peroxidase, or arylsulfotransferase, and thereby xenobiotic metabolism is promoted.

GST is an enzyme found largely in the liver, and conjugates various electrophilic compounds with reduced glutathione. GST catalyzes conjugation of metabolites exhibiting toxicity which are derived from metabolism by a phase I detoxifying enzyme, or of other xenobiotic substances (toxic substances), and thereby a detoxifying process is promoted. In other words, various disease risks caused by toxic substances can be decreased by enhancing GST activity in the living body. In recent years, substances capable of enhancing GST activity which are derived from natural products have been searched. For example, germacranoid which is contained in soybean processed foods or laurel, one or more plants selected from labiatae plants and myrtaceous Eucalyptus plants, or an extract thereof, limonoid glycosides, and the like has been known (for example, Patent Documents 1 to 4).

Glutathione is a tripeptide composed of cysteine, glutamic acid and glycine, and is widely distributed in the living body. Glutathione is a substrate for GST or glutathione peroxidase as described above, and therefore is an essential component for these enzymes to exhibit a detoxifying effect. In addition, glutathione also exhibits a detoxifying effect by non-enzymatically binding with various harmful substances.

QR is an enzyme catalyzing reduction of quinones or electron acceptor compounds in combination with NADH or NADPH as a coenzyme, and reduces and detoxifies oxides derived from metabolism by a phase I enzyme or oxides produced from active oxygen or lipid peroxide. In recent years, substances capable of enhancing QR activity which are derived from natural products have been searched. For example, sulfur-containing compounds such as isothiocyanate which is a component contained in Brassicaceae plants, and indirubin contained in indigo plants has been known to have QR activity enhancing effect (for example, Patent Document 5, Non-patent Document 1).

UGT is an enzyme that catalyzes a reaction (glucuronidation) for forming complexes (glucuronosides) of xenobiotic substances with glucuronic acid by transferring glucuronic acid to metabolites derived from metabolism by a phase I enzyme or other xenobiotic substances using UDP-glucuronic acid as a sugar donor. Thus, UGT is known to enhance water solubility of a substrate molecule to promote transfer of the substrate molecule into bile or blood, which leads to detoxification. In recent years, substances capable of enhancing UGT activity which are derived from natural products have been searched. For example, indigoid contained in indigo plants has been known to have UGT activity enhancing effect (For example, Patent Document 6).

Agar is a polysaccharide composed of agarose and agaropectin, and widely used as a food material. Agaro-oligosaccharides, which are low molecular compounds of agarose, have 3,6-anhydrogalactopyranose at the reducing end. Development of agaro-oligosaccharides as materials for health foods has been desired. It has been reported that agaro-oligosaccharides have physiological effects such as an anti-rheumatic effect and an anti-inflammatory effect (for example, Patent Documents 7 to 9).

In addition, L-glycero-1,5-epoxy-1αβ,6-dihydroxy-cis-hexa-3-en-2-one (DGE) is a compound obtained by keeping an agaro-oligosaccharide as described above under conditions from neural to alkali. It has been reported that DGE has physiological effects such as an anti-rheumatic effect and an anti-inflammatory effect (for example, Patent Document 10).

### Patent Document 1: JP-A 10-234326

### Patent Document 2: JP-A 9-234020

### Patent Document 3: JP-A 2006-111585

### Patent Document 4: JP-A 2000-316527

### Patent Document 5: JP-A 2003-40774

### Patent Document 6: JP-A 2003-246734

### Patent Document 7: WO 00/43018

### Patent Document 8: WO 99/24447

### Patent Document 9: WO 2003/086422

### Patent Document 10: WO 99/64424

Non-patent Document 1: Y. Zhang, et al., Proc Natl Acad Sci USA, 1992, Vol. 89, p2399-2403

### DISCLOSURE OF THE INVENTION

### Problems to be Solved by the Invention

An object of the present invention is to develop a substance having a detoxifying effect which can be safely and easily ingested and is suitable as a food material, a pharmaceutical material or a feed material, and thereby, to provide a drug, a food or a feed which utilizes the functionality of the substance.

### Means for Solving the Problems

As briefly described, the first aspect of the present invention relates to an enhancer of a phase II detoxifying enzyme activity or an intracellular glutathione content, which contains at least one compound selected from the group consisting of agar, agarose, a low molecular compound of agarose having 3,6-anhydrogalactopyranose at the reducing end, a compound represented by the following formula (Chemical Formula 1):

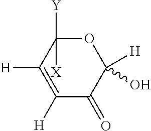

wherein X and Y are H or CH₂OH, provided that Y is H when X is CH₂OH, and Y is CH₂OH when X is H; a derivative thereof, and a salt thereof, as an active ingredient. In the first aspect of the present invention, examples of the low molecular compound of agarose having 3,6-anhydrogalactopyranose at the reducing end include agaro-oligosaccharides, particularly preferably, an agaro-oligosaccharide that is a mixture comprising agarobiose, agarotetraose, agarohexaose and agarooctaose. Further, in the first aspect of the present invention, examples of the phase II detoxifying enzyme include glutathione S-transferase, quinone reductase, and UDP-glucuronosyltransferase.

The second aspect of the present invention relates to a drug which contains the enhancer of a phase II detoxifying enzyme activity or an intracellular glutathione content according to the first aspect of the present invention.

The third aspect of the present invention relates to a food or feed which contains the enhancer of a phase II detoxifying enzyme activity or an intracellular glutathione content according to the first aspect of the present invention.

### EFFECT OF THE INVENTION

According to the present invention, there are provided an enhancer of a phase II detoxifying enzyme activity or an intracellular glutathione content, which contains at least one compound selected from the group consisting of agar, agarose, a low molecular compound of agarose having 3,6-anhydrogalactopyranose at the reducing end, a compound represented by the above-described formula (Chemical Formula 1), a derivative thereof and a salt thereof, as an active ingredient, and a drug, a food or a feed which contains the enhancer. The drug, food or feed which contains the enhancer promotes a detoxifying process by the effect of enhancing a phase II detoxifying enzyme activity or an intracellular glutathione content, and therefore, is extremely useful as a drug, a food or a beverage for treatment or prevention of various diseases, in particular, as a drug or a functional food for disease prevention which decreases risks of various diseases caused by toxic substances.

### BEST MODE FOR CARRYING OUT THE INVENTION

Examples of agar that can be used in the present invention include products obtained from red algae belonging to Gelidiaceae such as Gelidium amansii, Gelidium japonicum, Gelidium pacificum, Gelidium subcostatum, Pterocladia tenuis and Acanthopeltis japonica, red algae belonging to Gracilariaceae such as Gracilaria verrucosa and Gracilaria gigas, red algae belonging to Ceramiaceae such as Ceramium kondoi and Campylaephora hypnaeoides, as well as other red algae, as law materials. Usually, algae dried in the sun are used as the raw materials. Both fresh algae and dried algae can be used in the present invention. Algae that are bleached while spraying water during the drying, so-called bleached raw algae, can be also used. Raw material algae are extracted with hot water and then cooled to obtain so-called “Tokoroten (gelidium jelly)”. The “Tokoroten” is subjected to freeze-dehydration or compression-dehydration to remove water, and then dried to obtain agar. In the present invention, agar originated from various kinds of algae and agar in various forms including bar, belt, board, thread and powder forms can be used. Further, commercially available agar having various strengths can be used.

Agar normally contains about 70% of agarose and about 30% of agaropectin. As agarose in the present invention, agarose prepared from agar by a known purification method can be used. Purified agarose with low purity or high purity having various agarose contents can be used. In addition, commercially available agarose can be used.

In the present invention, agar and agarose are defined as those having a molecular weight of 10,000 or more. Agar and agarose having a molecular weight of less than 10,000 are defined as their low molecular compounds as described later. That is to say, agar or agarose which has undergone a degradation treatment such as an acid treatment but still has a molecular weight of 10,000 or more is included in the agar or agarose as used herein.

In the present invention, a low molecular compound of agarose having 3,6-anhydrogalactopyranose at the reducing end can be produced by partially degrading the above-described agar, agarose, or a sea alga that is the raw material of agar or agarose by a chemical, physical and/or enzymatic method. The chemical, physical and/or enzymatic method for partial degradation is not particularly limited as long as a low molecular compound having 3,6-anhydrogalactopyranose at the reducing end is obtained. An example of the chemical degradation method includes hydrolysis under acid to neutral conditions. An example of the physical degradation method includes cutting and degradation by irradiation of electromagnetic waves or ultrasonic waves. An example of the enzymatic degradation method includes hydrolysis with a hydrolyzing enzyme such as agarase. Particularly preferred examples of the degradation method include acid degradation and enzymatic degradation with α-agarase, from the viewpoint of efficient production of an agarose low molecular compound having 3,6-anhydrogalactopyranose at the reducing end.

In the present invention, examples of the low molecular compound having 3,6-anhydrogalactopyranose at the reducing end include agarose low molecular compounds having a molecular weight of less than 10,000 and composed of preferably 2 to 50 sugars, more preferably 2 to 30 sugars in which β-D-galactose and 3,6-anhydrogalactopyranose are alternately arranged. Particularly preferred examples thereof include agaro-oligosaccharides. As used herein, the agaro-oligosaccharides mean agarobiose, agarotetraose, agarohexaose, agarooctaose, and mixtures of two or more kinds selected from agarobiose, agarotetraose, agarohexaose and agarooctaose, and therefore, are distinguished from neoagaro-oligosaccharides that have β-D-galactose at the reducing end.

As the agaro-oligosaccharide used in the present invention, agarobiose, agarotetraose, agarohexaose or agarooctaose may be used alone, and a mixture thereof is preferably used. When an agaro-oligosaccharide containing agarobiose, agarotetraose, agarohexaose and agarooctaose is used in the present invention, the agaro-oligosaccharide can be produced by a known production method including, but not limited to, a production method described in WO 00/69285. That is, an agaro-oligosaccharide containing agarobiose, agarotetraose, agarohexaose and agarooctaose which is obtained from a raw material agar by acid degradation with a solid acid can be used in the present invention. A commercially available agaro-oligosaccharide containing agarobiose, agarotetraose, agarohexaose and agarooctaose (product name: Agaoligo, manufactured by Takara Bio Inc.) can be also used.

The compound represented by the above formula (Chemical Formula 1) used in the present invention can be obtained by keeping a compound having 3,6-anhydrogalactopyranose at the reducing end, or the like under neutral to alkaline conditions. The compound represented by the above formula (Chemical Formula 1) can be also obtained by subjecting a compound containing 3,6-anhydrogalactopyranose in its structure to acid hydrolysis and/or enzymatic degradation at a pH of less than 7, and then keeping the acid degraded and/or enzymatically degraded compound thus obtained under neutral to alkaline conditions. Examples of the compound containing 3,6-anhydrogalactopyranose at the reducing end include agaro-oligosaccharides such as agarobiose, agarotetraose, agarohexaose and agarooctaose, and K-carrabiose. Examples of the compound having 3,6-anhydrogalactopyranose in its structure include agar, agarose, their degradation products, and the low molecular compounds of agarose having 3,6-anhydrogalactopyranose at the reducing end as described above.

When the compound having 3,6-anhydrogalactopyranose at the reducing end, such as agarobiose or K-carrabiose, is kept under neutral to alkaline conditions at a pH of 7 or more, a solution or suspension of at least one compound selected from the above-described compounds having 3,6-anhydrogalactopyranose at the reducing end is used for the reaction, and the composition of such a reaction liquid used for carrying out the reaction is not particularly limited. Preferably, a reaction liquid containing an alkali including, but not limited to, an inorganic base such as sodium hydroxide, potassium hydroxide, calcium hydroxide or ammonia, and an organic base such as Tris, ethylamine or triethylamine, dissolved in water (for example, distilled water, ion exchange water, tap water etc.) as a solvent can be used. The alkali concentration is not particularly limited. A reaction liquid with an alkali concentration of preferably 0.0001 to 5 N, more preferably 0.001 to 1 N can be used. The reaction temperature is not particularly limited, and may be preferably 0 to 200° C., more preferably 20 to 130° C. The reaction time is not particularly limited, and may be preferably for several seconds to several days. The kind and concentration of an alkali, the reaction temperature and the reaction time, as well as the amount of the compound as a raw material to be dissolved or suspended in a reaction liquid may be appropriately selected depending on the kind of the compound and the production amount of the desired compound represented by the above-described formula (Chemical Formula 1). The reaction liquid usually may have a pH of 7 or more. However, production of the compound represented by the above-described formula (Chemical Formula 1) rapidly proceeds when the reaction liquid has a higher alkali concentration as compared with a lower alkali concentration and when the reaction temperature is a higher temperature as compared with a lower temperature. For example, the compound represented by the above-described formula (Chemical Formula 1) is produced by preparing a solution with pH 11.5 of agarobiose or K-carrabiose and keeping the solution at 37° C. for 5 minutes.

An alkali solution containing the compound represented by the above-described formula (Chemical Formula 1) thus produced may be used after neutralization or may be used as an acid solution after adjustment of pH to less than 7, depending on purposes. When the compound containing 3,6-anhydrogalactopyranose in its structure is subjected to acid hydrolysis and/or enzymatic degradation at a pH of less than 7, and then kept under neutral to alkaline conditions in the same manner as described above to obtain the compound represented by the above-described formula (Chemical Formula 1), the acid hydrolysis may be carried out, for example, by preparing a reaction liquid using water as a solvent and preferably 0.001 to 5 N of an acid including an inorganic acid such as hydrochloric acid, sulfuric acid or nitric acid, and an organic acid such as citric acid, formic acid, acetic acid, lactic acid or ascorbic acid, dissolving or suspending a suitable amount of a raw material compound in the reaction liquid, and then keeping the reaction liquid at a reaction temperature of preferably 0 to 200° C. for a reaction time of preferably several seconds to several days. A solid acid can also be used as the acid. In the case of the enzymatic degradation, the reaction may be carried out in the same reaction liquid and under the same reaction conditions as used for the acid hydrolysis, for example, using a suitable amount of α-agarase, for example, α-agarase described in WO 00/50578 as an enzyme under conditions where the enzyme shows activity.

The compound represented by the above-described formula (Chemical Formula 1) of the present invention contained in the reaction liquid may be purified by a known purification means including chemical methods and physical methods. The compound may be purified by a combination of purification methods including a gel filtration method, a fractionation method using a molecular weight-fractioning membrane, a solvent extraction method, and various chromatography methods using an ion exchange resin and the like. For example, the compound represented by the above-described formula (Chemical Formula 1) wherein X is CH₂OH and Y is H, L-glycero-1,5-epoxy-1αβ, 6-dihydroxy-cis-hexa-3-en-2-one (hereinafter sometimes, referred to as DGE) is purified from a product obtained by a treatment of agarobiose under neural to alkaline conditions. The compound represented by the above-described formula (Chemical Formula 1) wherein X is H and Y is CH₂OH, D-glycero-1,5-epoxy-1αβ, 6-dihydroxy-cis-hexa-3-en-2-one (hereinafter sometimes, referred to as K-DGE) may be purified from a product obtained by a treatment of K-carrabiose under neutral to alkaline conditions. Herein, DGE is believed to be a compound produced when an agaro-oligosaccharides as described above is taken into the living body [Jpn. J. Phycol. (Sorui) 48: 13-19, Mar. 10, 2000]. The structure of DGE is shown as the following formula (Chemical Formula 2).

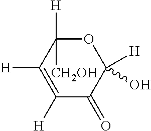

Further, derivatives of the above-descried compounds can be used as the active ingredient in the present invention. Examples of the derivatives include the compounds to which various substituents are bound. However, the derivatives are not particularly limited, as long as they can exert the desired effects. Examples of the substituent include aliphatic groups (linear aliphatic groups such as a methyl group, an ethyl group and an n-propyl group, and branched chain aliphatic groups such as an isopropyl group, an isobutyl group, a prenyl group and a geranyl group), aromatic groups (such as a phenyl group, a naphthyl group, a biphenyl group, a pyrrolyl group and an indolyl group), aromatic aliphatic groups (such as a benzyl group and a phenethyl group), a hydroxyl group, a carboxyl group, a sulfate group, a phosphate group, a thiol group, an amino group, a nitro group, an alkoxy group (such as a methoxy group), an acyloxy group (such as an acetyl group), halogens (such as chlorine, bromine, and fluorine), amino acids, and peptides. In addition, the derivative may be a derivative of the compound which can function as a prodrug, as described later.

As a derivative of the active ingredient of the present invention, examples of a derivative of agar, agarose, or a low molecular compound of agarose having 3,6-anhydrogalactopyranose at the reducing end such as an agaro-oligosaccharide include, but not particularly limited to, preferably a sulfated product and a methylated product. A preferred example of a derivative of the compound represented by the above-described formula (Chemical Formula 1) is a derivative produced by a reaction of the compound with a SH group-containing compound. The structure of such a derivative is shown as the following formula (Chemical Formula 3).

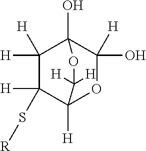

wherein R is a residue obtained by removing a SH group from a SH group-containing compound.

The SH group-containing compound to be used is not particularly limited as long as it has at least one SH group. In the above-described formula (Chemical Formula 3), R is a residue remaining after one SH group is consumed by a binding of the compound represented by the above-described formula (Chemical Formula 1) with the SH group-containing compound in a reaction of the SH group-containing compound and the compound represented by the above-described formula (Chemical Formula 1). Therefore, when the SH group-containing compound has 2 or more of SH groups, 1 or more of SH groups are present in the residue represented by R. Examples of the SH group-containing compound include methanethiol, butanethiol, mercaptoethanol, SH group-containing amino acids, and SH group-containing amino acid derivatives.

Examples of the SH group-containing amino acid include cysteine and homocysteine. Examples of the SH group-containing amino acid derivative include derivatives of the above-described amino acids, such as cysteine derivatives, cysteine-containing peptides and cysteine derivative-containing peptides. Examples of the cysteine derivative include amide compounds, acetyl compounds and ester compounds of cysteine. The cysteine-containing peptide is not particularly limited as long as it has cysteine as a constituent component in the peptide. The cysteine-containing peptides include oligopeptides, low molecular peptides such as glutathione, and high molecular peptides made of polypeptides such as a protein. Further, a peptide containing cystine or homocystine can also be used as the cysteine- or homocysteine-containing peptide in the present invention by combining the above-described reaction with the condition capable of changing a cystine- or homocystine-containing peptide to a cysteine- or homocysteine-containing peptide, for example, a reduction treatment. Examples of the cysteine derivative-containing peptide include substances which are the same as the above-described cysteine-containing peptides except that they contain cysteine derivatives in place of cysteine. Examples of the cysteine-containing peptide also include cysteine-containing peptides containing carbohydrate, lipid, and the like. Further, salts, acid anhydrides, esters and the like of the above-described various substances may be used.

Production of a compound represented by the above-described formula (Chemical Formula 3) is not particularly limited, and, for example, can be performed according to a method described in WO 99/64424.

A salt of the above-described compound to be used in the present invention is preferably a pharmaceutically acceptable salt, and can be obtained by a known conversion method. Examples of the salt include salts formed with inorganic acids such as hydrochloric acid, hydrobromic acid, hydroiodic acid and sulfuric acid, and salts with organic acids such as formic acid, acetic acid, oxalic acid, malonic acid and succinic acid, and ammonium salts obtained by reaction with alkyl halide such as methyl iodide, benzyl halide, and the like.

Furthermore, the compound used in the present invention can form a derivative (prodrug) which can be easily hydrolyzed in the body to exert the desired effects, and, for example, the compound cab be esterified. Preparation of such a prodrug may be performed according to a known method.

In addition, as the active ingredient of the present invention, various isomers such as optical isomers, keto-enol tautomeric isomers and geometric isomers of the above-described compounds can be also used. Further, the active ingredient may be also a separated isomer or a mixture of isomers.

The present invention provides an enhancer of a phase II detoxifying enzyme activity or an intracellular glutathione content (hereinafter sometimes, referred to as the enhancer of a phase II detoxifying enzyme activity or an intracellular glutathione content of the present invention), which contains at least one compound selected from the group consisting of agar, agarose, an agarose low molecular compound having 3,6-anhydrogalactopyranose at the reducing end, a compound represented by the above-described formula (Chemical Formula 1), a derivative thereof and a salt thereof, as an active ingredient (hereinafter sometimes, referred to as the active ingredient of the present invention).

Examples of the phase II detoxifying enzyme as used herein include glutathione S-transferase (GST), quinone oxidase (QR), UDP-glucuronosyltransferase (UGT), glutathioneperoxidase, and arylsulfotrasferase. Particularly preferred examples are GST, QR and UGT. The enhancing effect on the activity of GST, QR or UGT produced by the active ingredient of the present invention can be evaluated by, for example, but not particularly limited to, measurement of the enzyme activity of GST, QR or UGT or measurement of the gene expression amount of GST, QR or UGT, as shown in Examples 1 to 7 described later.

The enhancing effect on an intracellular glutathione content produced by the active ingredient of the present invention can be evaluated by, for example, but not particularly limited to, measurement of the glutathione content in a cell as shown in Example 8 described later.

The enhancer of a phase II detoxifying enzyme activity or an intracellular glutathione content of the present invention enhances the activity of the phase II detoxifying enzymes as described above such as GST, QR and UGT, and further enhances the amount of glutathione, which can be a substrate for some phase II detoxifying enzymes, contained in cells and thereby can promote toxic substance metabolism in the living body, particularly in the liver, and the function of liver. As a result, the enhancer of a phase II detoxifying enzyme activity or an intracellular glutathione content of the present invention can decrease risks of various diseases caused by various toxic substances, and therefore are extremely suitable for use in drugs and functional foods as described later. Toxic substances whose metabolism can be promoted by GST, QR, UGT and glutathione are not particularly limited to specific compounds, and include wide variety of exogenous substances such as carcinogenic substances, agrichemicals, environmental pollutants, and drugs having side effects. That is, diseases on which the enhancer of a phase II detoxifying enzyme activity or an intracellular glutathione content of the present invention has the effect are not limited to specific diseases. In addition, such an enhancing effect on a phase II detoxifying enzyme activity produced by the active ingredient of the present invention is not found in neoagaro-oligosaccharides having β-D-galactose at the reducing end, as shown in Comparative Example described later.

According to the present invention, a drug which contains the enhancer of a phase II detoxifying enzyme activity or an intracellular glutathione content of the present invention (hereinafter sometimes, referred to as the drug of the present invention) is provided. The drug of the present invention can promote the hepatic function for metabolism of toxic substances by enhancing the activity of a phase II detoxifying enzyme or enhancing the glutathione content in cells. The drug of the present invention is useful for treatment or prevention of various diseases accompanied with deterioration in liver function, such as liver inflammation, liver cirrhosis, liver cancer, fatty liver, and alcoholic liver disease. The drug of the present invention is extremely suitable as a preventive drug for decreasing risks of developing these diseases particularly due to the enhancing effect on a phase II detoxifying enzyme activity or an intracellular glutathione content produced by the active ingredient of the present invention. Further, when the drug of the present invention is used in combination with a drug that causes liver damage as a side effect, the drug of the present invention can decrease liver damage caused by drug administration due to the toxic substance metabolism promoting effect of the drug of the present invention. In this case, the active ingredient of the present invention and the other drug may be mixed to be formulated into a dosage form, or may be separately formulated into separate dosage forms and then simultaneously administered.

In addition, the drug of the present invention is useful for prevention or treatment of diseases caused by various toxic substances, in addition to the above-described various diseases. Examples of such diseases include, but not particularly limited to, cancer, arteriosclerosis, Alzheimer's disease, obesity (metabolic syndrome), and dermatosis.

Examples of the drug of the present invention include formulations obtained by mixing the above-described active ingredient used as the enhancer of a phase II detoxifying enzyme activity or an intracellular glutathione content of the present invention with a known pharmaceutical carrier.

As used herein, the drug includes quasi drugs. Further, the drug of the present invention can be also used in combination with other ingredients usable for the same purposes as those of the active ingredient of the present invention, that is, phase II detoxifying enzymes or other ingredients that are known to have an enhancing effect on a phase II detoxifying enzyme activity. Examples of the phase II detoxifying enzyme are the same as the above-described phase II detoxifying enzymes. Examples of the other ingredients that are known to have an enhancing effect on a phase II detoxifying enzyme activity include, but not particularly limited to, isothiocyanate and curcumin.

The drug of the present invention can also be used in combination with glutathione or an ingredient containing a large amount of glutathione. The detoxifying effect of the drug of the present invention can be further enhanced by adding glutathione that can be a substrate for a phase II detoxifying enzyme whose activity is enhanced by the active ingredient of the present invention, or an ingredient capable of increasing intracellular glutathione. Examples of the ingredient capable of increasing intercellular glutathione include, but not particularly limited to, isothiocyanate.

The drug of the present invention can be usually produced by mixing the above-described active ingredient with a pharmaceutically acceptable liquid or solid carrier, and optionally adding a solvent, a dispenser, an emulsifier, a buffer, a stabilizer, an excipinet, a binder, a disintegrant, a lubricant, and the like, to formulate the mixture into a solid dosage form such as a tablet, a granule, a powder, a particulate agent or a capsule, or a liquid dosage form such as a conventional liquid agent, a suspension or an emulsion. In addition, the drug of the present invention may be formulated into a dried product for reconstruction in the liquid state with a suitable vehicle before use, or an external preparation.

A pharmaceutical carrier can be selected according to the administration mode and the dosage form of a drug. In the case where the drug of the present invention is an oral drug of a solid composition, examples of the oral drug of a solid composition include a tablet, a pill, a capsule, a powder, a fine granule and a granule, and examples of a pharmaceutical carrier that can be used include starch, lactose, sucrose, mannite, carboxymethylcellulose, corn starch, and inorganic salts. In preparation of the oral drug, a binder, a disintegrant, a surfactant, a lubricant, a fluidity accelerator, a corrigent, a colorant, a perfume and the like may be added. For example, when the oral drug is a tablet or a pill, the oral drug may be coated with a sugar coating or an enteric- or gastric-soluble film which is made of sucrose, gelatin, hydroxypropylcellulose or the like, as desired. In the case where the drug of the present invention is an oral drug of a liquid composition, examples of the oral drug of a liquid composition include a pharmacologically acceptable emulsion, a solution, a suspension and a syrup, and examples of a pharmaceutical carrier that can be used include purified water and ethanol. In preparation of the oral drug of a liquid composition, an auxiliary agent such as a wetting agent or a suspending agent, a sweetener, a flavor, an antiseptic agent, and the like may be further added as desired.

In the case where the drug of the present invention is a parenteral drug, it can be prepared by dissolving or suspending the above-described active ingredient of the present invention in a diluent such as distillation water for injection, saline, an aqueous glucose solution, a vegetable oil for injection, a sesame oil, a peanut oil, a soybean oil, a corn oil, propylene glycol or polyethylene glycol, and optionally adding thereto a bacteriocide, a stabilizer, a tonicity agent, a soothing agent, and the like. The parenteral drug may be also produced as a solid composition which can be dissolved in sterile water or a sterile solvent for injection before use.

Examples of the external preparation include solid, semi-solid, or liquid preparations for transdermal administration or transmucosal (buccal or intranasal) administration. Further, suppository and the like are included. Specific examples of the external preparation include an emulsion such as an emulsified agent or a lotion, a liquid preparation such as an external tincture or a liquid agent for transmucosal administration, an ointment such as an oily ointment or a hydrophilic ointment, and a patch for transdermal administration or transmucosal administration such as a film, a tape or a compress.

The above-described various dosage forms of the drug can be appropriately produced using a known pharmaceutical carrier and the like by a conventional method. The content of the active ingredient in the drug is varied depending on a dosage form, an administration method and the like, and is not particularly limited. Preferably, the content of the active ingredient in the drug is such an amount that the active ingredient can be administered in the range of an administration amount as described later. The content of the active ingredient in the drug of the present invention is usually about 1 to 100% by weight.

The drug of the present invention is administered by an administration method suitable for its dosage form. The administration method is not particularly limited. For example, the drug of the present invention can be administered internally, externally, or by an injection. When a therapeutic drug or a preventive drug of the present invention is administered by an injection, it may be administered intravenously, intramuscularly, subcutaneously, intradermally or the like. When the drug of the present invention is administered externally, it may be administered by a suitable administration method, for example, as an external preparation such as suppository.

A dose of the drug of the present invention is appropriately selected depending on a dosage form, an administration method and an intended use thereof, and the age, weight and symptom of a subject patient for administration of the drug, and thus it is not fixed. Usually, a dose of the active ingredient contained in the drug is preferably 0.005 to 5,000 mg/kg body weight, more preferably 0.05 to 500 mg/kg body weight, further more preferably 0.5 to 50 mg/kg body weight per day for an adult. Of course, the dose varies depending on various conditions, and thus, may be smaller than the above-described dose range or may exceed the above-described dose range. Administration may be carried out once a day or several times a day within the desired dose range. An administration term is arbitrary. The drug of the present invention can be orally administrated as it is, or can be also added to any food which is ingested every day.

According to the present invention, a food or feed which contains the enhancer of a phase II detoxifying enzyme activity or an intracellular glutathione content of the present invention (hereinafter sometimes, referred to as the food or feed of the present invention) is provided. The food or the feed of the present invention can enhance the activity of a phase II detoxifying enzyme or the glutathione content in cells, in the same manner as the drug of the present invention, and therefore, can be used as a food or feed for enhancing a phase II detoxifying enzyme activity or an intracellular glutathione content. The food or feed of the present invention is useful for treatment or prevention of various diseases accompanied with deterioration in liver function, such as liver inflammation, liver cirrhosis, liver cancer, fatty liver, and alcoholic liver disease. The food or feed of the present invention is extremely suitable as a functional food for decreasing risks of developing these diseases particularly due to the enhancing effect on a phase II detoxifying enzyme activity or an intracellular glutathione content which is produced by the active ingredient of the present invention.

In addition, the food or feed of the present invention is useful for prevention or treatment of diseases caused by toxic substances accumulated in the living body, in addition to the above-described various diseases. Examples of such diseases include, but not particularly limited to, cancer, arteriosclerosis, Alzheimer's disease, obesity (metabolic syndrome), and dermatosis. Further, ingestion of the food or feed of the present invention can result in amelioration of deconditioning such as skin roughness and fatigue due to its detoxifying effect.

As one aspect of the food of the present invention, there is a food having a detoxifying effect, that is, a detox effect. Examples of such a food include a functional food for decreasing risks of developing the above-described diseases (a food for health maintenance), a functional food for antiaging that promotes detoxification in the living body to prevent aging phenomena, and a functional food for hangover prevention that is taken before or after alcohol intake. The functional food includes a specified health food with a label indicating that the active ingredient of the present invention is involved in the function of the food, and the food decreases risks of developing the above-described diseases, has a detoxifying effect, has an antiaging effect or has a hangover prevention effect.

The food or feed of the present invention, in the same manner as the drug of the present invention, can be also used in admixture with other ingredients usable for the same purposes as those of the active ingredient of the present invention, that is, phase II detoxifying enzymes or other ingredients that are known to have an enhancing effect on a phase II detoxifying enzyme activity. Furthermore, the food or feed of the present invention can be also used in admixture with phase I detoxifying enzymes or ingredients that are known to have an enhancing effect on its activity, in the same manner as the drug of the present invention. Furthermore, the food or feed of the present invention can be also used in admixture with glutathione or an ingredient containing a large amount of glutathione. It is preferable that the food of the present invention is mixed with ingredients preferably usable as food materials among the above-described ingredients that can be mixed, such as broccoli, turmeric, zedoary, yeast, freshwater clam extract, oyster extract, milk thistle extract, fucoidan, Angelica keiskei, and their processed products.

The term “contain” referring to the food or feed of the present invention means containing, adding and/or diluting. Herein, the term “containing” indicates that the active ingredient used in the present invention is contained in a food or a feed. The term “adding” indicates that the active ingredient used in the present invention is added to raw materials of a food or a feed. The term “diluting” indicates that raw materials of a food or a feed are added to the active ingredient used in the present invention. The food of the present invention also includes a food product to which the active ingredient is added as a food additive.

A method for producing the food or feed of the present invention is not particularly limited as long as the active ingredient of the present invention can be contained in the obtained food or feed. For example, mixing, cooking, processing and the like may be performed according to those for usual foods or feeds. The food or feed of the present invention can be produced by a production method for usual foods or feeds.

Examples of the food of the present invention include, but not particularly limited to, products of processed cereal (e.g., wheat flour product, starch product, premixed product, noodle, macaroni, bread, bean jam, buckwheat noodle, wheat-gluten bread, rice noodle, gelatin noodle and packed rice cake), products of processed fat and oil (e.g., plastic fat and oil, tempura oil, salad oil, mayonnaise and dressing), products of processed soybeans (e.g., tofu, miso and fermented soybean), products of processed meat (e.g., ham, bacon, pressed ham and sausage), processed marine products (e.g., frozen ground fish, boiled fish paste, tubular roll of boiled fish paste, cake of ground fish, deep-fried patty of fish paste, fish ball, sinew, fish meat ham or sausage, dried bonito, product of processed fish egg, canned marine product and fish boiled in sweetened soy sauce), dairy products (e.g., raw milk, cream, yogurt, butter, cheese, condensed milk, powdered milk and ice cream), products of processed vegetables and fruits (e.g., paste, jam, pickle, fruit juice, vegetable drink and mixed drink), confectioneries (e.g., chocolate, biscuit, sweet bun, cake, rice-cake sweet, and rice sweet), alcoholic drinks (e.g., sake, Chinese liquor, wine, whisky, shochu, vodka, brandy, gin, rum, beer, soft alcoholic drink, fruit liquor and liqueur), luxury drinks (e.g., green tea, tea, oolong tea, coffee, soft drink and lactic acid drink), seasonings (e.g., soy sauce, sauce, vinegar, and sweet sake), canned, bottled or bagged foods (e.g., various cooked foods such as rice topped with cooked beef and vegetables, rice boiled together with meat and vegetables in a small pot, steamed rice with red beans, and curry), semi-dried or condensed foods (e.g., liver paste, other spreads, soup for buckwheat noodle or udon and condensed soup), dried foods (e.g., instant noodle, instant curry, instant coffee, powdered juice, powdered soup, instant miso soup, cooked food, cooked drink and cooked soup), frozen foods (e.g., sukiyaki, chawan-mushi, grilled eel, hamburger steak, shao-mai, dumpling stuffed with minced pork, various stick-shaped foods and fruit cocktail), solid foods, liquid foods (e.g., soup), processed agricultural or forest products (e.g., spice), processed livestock products, and processed marine products, which contain the active ingredient of the present invention. As used herein, foods include beverages. For example, a beverage according to the present invention can be produced by dissolving an agaro-oligosaccharide in water and appropriately adding ingredients used in existing beverages thereto.

The food of the present invention may be in any form including orally ingestible forms such as a powdery form, a tablet form, a granular form, and a capsulate form, as long as one or more the active ingredients are contained, added and/or diluted in the food of the present invention and the content thereof corresponds to an amount necessary for exhibiting the enhancing effect on a phase II detoxifying enzyme activity or an intracellular glutathione. The food of the present invention also includes the above-described active ingredient of the present invention as it is, and a mixture of the active ingredient with a suitable emulsifier, excipient or the like at an appropriate proportion. These foods can be eaten as they are, or can be mixed with water and then ingested as beverages.

The content of the active ingredient in the food of the present invention is not particularly limited, and can be suitably selected in view of functionality and activity exhibition thereof. For example, the content of the active ingredient in the food of the present invention is preferably 0.0001 to 100% by weight, more preferably 0.001 to 60% by weight, further more preferably 0.01 to 30% by weight.

The food of the present invention may be ingested in such an amount that the active ingredient of the present invention can be ingested in an amount of preferably 0.005 to 5,000 mg/kg body weight, more preferably 0.05 to 500 mg/kg body weight, further more preferably 0.5 to 50 mg/kg body weight per day for an adult.

Further, the present invention provides a feed for organisms having the enhancing effect on a phase II detoxifying enzyme activity or an intracellular glutathione content, which contains, that is, contains, adds and/or dilutes the above-described active ingredient. As another aspect of the present invention, there is provided a method of rearing an organism which comprises administering the above-described active ingredient to the organism. As a further another aspect of the present invention, there is provided an organism rearing agent which contains the above-described active ingredient.

Examples of the organism as used herein include, but not particularly limited to, bred animals and pet animals. Examples of the bred animals include livestock such as a horse, cattle, a pig, a sheep, a goat, a camel and a lama, experimental animals such as a mouse, a rat, a guinea pig and a rabbit, poultry such as a chicken, a duck, a turkey and an ostrich, fishes, crustacean, and shells. Examples of the pet animals include dogs and cats. As a feed, feeds for keeping and/or improving physical conditions are exemplified. As the organism rearing agent, an immersion agent, a feed additive, and a beverage additive are exemplified.

According to these inventions, it can be expected that the same effects as the drug of the present invention is exhibited in the above-described organisms to which these inventions are applied, based on the enhancing effect on a phase II detoxifying enzyme activity or an intracellular glutathione of the active ingredient of the present invention. That is, the feed of the present invention can treat or prevent various diseases caused by toxic substances in the organisms, and for example, can decrease risks of developing various diseases caused by toxic substances.

The above-described active ingredient used in the present invention is usually administered in an amount of 0.005 to 5,000 mg/kg body weight, more preferably 0.05 to 500 mg/kg body weight, further more preferably 0.5 to 50 mg/kg body weight per day for a subject organism. The administration can be attained, for example, by adding and mixing the active ingredient in raw materials of an artificial blended feed to be given to a subject organism, or by mixing the active ingredient with powdery raw materials of the artificial blended feed and then further adding and mixing the mixture to other raw materials. The content of the active ingredient in the feed is not particularly limited, and may be appropriately selected depending on intended purposes. For example, the content of the active ingredient in the feed is preferably 0.0001 to 100% by weight, more preferably 0.001 to 60% by weight, further more preferably 0.01 to 30% by weight.

A method for producing the feed of the present invention is not particularly limited and blending thereof may be also in accordance with usual feeds, as long as the above-described active ingredient of the present invention is contained in the produced feed. The organism rearing agent can also be prepared in the same manner.

According to the present invention, physical conditions of livestock animals, experimental animals, poultry, pet animals and the like can be maintained in good conditions or can be improved, for example, by allowing a subject organism to ingest a feed which comprises the above-described active ingredient having an enhancing effect on a phase II detoxifying enzyme activity or an increasing effect on an intracellular glutathione which is used in the present invention, or immersing a subject organism in a liquid containing the above-described active ingredient used in the present invention (for example, a liquid obtained by dissolving the immersion agent in water). These aspects are a part of the aspect of a rearing method of organisms in the present invention.

The above-described active ingredient used in the present invention is not found to have toxicity when it is administered in an effective amount for exhibiting its effect to the living body. For example, in the case of oral administration, there is no fatal case when agarobiose, agarotetraose, agarohexaose, agarooctaose, a mixture thereof, or DGE is administered to a mouse in an amount of 2,000 mg/kg body weight as a single dose. Further, there is no fatal case when the above-described active ingredient is administered orally as a single dose of 2,000 mg/kg body weight to a rat.

### EXAMPLES

The present invention will be more specifically described by reference to the following Examples which the present invention is not limited to. In Examples, “%” means “% by volume” unless otherwise stated.

### Preparation Example 1

### Preparation of Agarobiose

Agar (AGAR NOBLE) was suspended in 0.1 N of HCl so as to have a concentration of 10%, and then heated at 100° C. for 19 minutes. To a Toyopearl HW40C (manufactured by TOSOH CORPORATION) column (4.4 cm×85 cm) equilibrated with water, 10 ml of the above sample was applied. Gel filtration chromatography was carried out using water as a mobile phase at a flow rate of 1.4 ml per minute. A substance eluted was detected with a differential refractometer, and 7 ml of fractions were collected.

There were peaks at elution times of 406 minutes, 435 minutes, 471 minutes and 524 minutes. A fraction corresponding to each peak was spotted onto a silica gel 60 sheet F₂₅₄ (manufactured by Merck Co.), developed with 1 butanol:ethanol:water=5:5:1, and then analyzed by an orcinol-sulfuric acid method. As a result, it was found that the peak at 524 minutes was agarobiose. This fraction was freeze dried to obtain 140 mg of agarobiose.

### Preparation Example 2

### Preparation of L-glycero-1,5-epoxy-1αβ,6-dihydroxy-cis-hexa-3-en-2-one (DGE)

A suspension of 2.5 g of commercially available agar (AGAR NOBLE) in 50 ml of 0.1 N HCl was heated at 100° C. for 13 minutes to obtain a solution. The solution was cooled to room temperature, adjusted with NaOH to pH 12, and then neutralized.

The neutralized product was subjected to the following normal phase HPLC. Each peak was collected, dried under reduced pressure and then dissolved in water. A cancer cell growth suppressing activity of each fraction was measured using a HL-60 cell. It was found that a fraction at a retention time of 4.05 to 4.16 minutes had a cancer cell growth suppressing activity.

Then, the fraction at a retention time of 4.05 to 4.16 minutes was collected in large amounts, and subjected to structural analysis. As a result, it was found that the fraction was L-glycero-1,5-epoxy-1αβ, 6-dihydroxy-cis-hexa-3-en-2-one (DGE). Conditions used for the normal phase HPLC are shown below.

Column: PALPAK Type S (4.6 mm×250 mm, manufactured by TAKARA SHUZO CO., LTD.)
Mobile phase A: aqueous 90% acetonitrile solution
Mobile phase B: aqueous 50% acetonitrile solution
Flow rate: 1 ml/min
Elution: mobile phase A (10 minutes) → linear concentration gradient from mobile phase A to mobile phase B (40 minutes) → mobile phase B (10 minutes)
Detection: absorbance at 195 nm
Column temperature: 40° C.

### Example 1

### Evaluation (1) of Enhancing Effect on Glutathione S-transferase (GST) Activity

Hepa1c1c7 cells (ATCC CRL-2026) were suspended in a Dulbecco-modified Eagle medium (manufactured by Sigma) containing 10% of fetal bovine serum (manufactured by MP Biomedicals Co.) and 1% of penicillin-streptomycin (manufactured by Nacalai Tesque, Inc.) at 4×10⁵ cells/ml. To each well of a 96 well-microtiter plate, 0.2 ml of the cell suspension was added, and cultured at 37° C. overnight in the presence of 5% carbon dioxide gas. Then, the medium in the wells was replaced with a Dulbecco-modified Eagle medium. To each well, 0.4 μl of a solution of a test substance in water was added and then cultured for 24 hours. The agarobiose obtained in Preparation Example 1 and a commercially available agaro-oligosaccharide (trade name: Agaoligo, manufactured by Takara Bio Inc., containing agarobiose, agarotetraose, agarohexaose and agarooctaose in 20 to 25% each) were used as test substances. For a negative control, water was added in place of a test substance. After completion of the culture, the medium was removed and the cells were washed with a phosphate buffer. Then, 0.1 ml of a cell-lysis solution (10 mM Tris-HCl (pH 7.4), 38.5 mM KCl, and 1 mM EDTA, 1% NP-40) was added and incubated at 37° C. for 10 minutes to obtain an enzyme solution. To 25 μl of the enzyme solution, 155 μl of a reaction solution (0.13 M potassium phosphate buffer solution (pH 6.5), 1.3 mM glutathione) was added. Immediately before measurement, 20 al of 10 mM of CDNB (2,4-dinitrochlorobenzene, manufactured by TOKYO CHEMICAL INDUSTRY CO., LTD.) as a reaction substrate was added, and a change in an absorbance at 340 nm was measured. This activity assay was performed in triplicate. The protein content was measured by using a 50-fold dilution of the enzyme solution with a phosphate buffer and MicroBCA protein assay kit (manufactured by PIERCE Co.). The test substances were added in such amounts that they reached the concentrations shown in the following table. The GST activity was expressed as a GST activity relative to the control and calculated by the following formula.

GST activity=(Maximum rate coefficient in test substance-added section/Protein content in test substance-added section)/(Maximum rate coefficient in water-added section/Protein content in water-added section)

Results are shown in Table 1. Table 1 shows the GST activity in cells to which each test substance was added. It was found that the addition of an agaro-oligosaccharide or agarobiose resulted in a significant increase of the GST activity.

TABLE 1

| | | GST activity |
|---|---|---|
| Test substance | Final concentration | (−fold) |
| Agaro-oligosaccharide | 25 μg/ml | 1.2 |
| | 50 μg/ml | 1.7 |
| | 100 μg/ml | 1.8 |
| | 200 μg/ml | 1.8 |
| Agarobiose | 25 μM | 1.4 |
| | 50 μM | 1.5 |
| | 100 μM | 1.9 |
| | 200 μM | 1.9 |

### Example 2

### Evaluation (2) of Enhancing Effect on Glutathione S-transferase (GST) Activity

An effect on a GST activity caused by using DGE obtained in Preparation Example 2 was determined in accordance with Example 1. Each measurement was repeated three times. The GST activity was expressed as a GST activity relative to the control and calculated by the same formula as in Example 1.

Results are shown in Table 2. Table 2 shows the GST activity in cells to which DGE was added. It was found that the addition of DGE resulted in a significant increase of the GST activity.

TABLE 2

| Test substance | Final concentration | GST activity (−fold) |
|---|---|---|
| DGE | 5 μM | 1.4 |
| | 10 μM | 1.4 |
| | 20 μM | 1.6 |

### Example 3

### Measurement of Quinone Reductase (QR) Activity

A QR activity was measured by a partially modified method from a method described in Hans J. Prochaska et al., Analytical Biochemistry 169, 328-336 (1988). Hepa1c1c7 cells were suspended in a Dulbecco-modified Eagle medium containing 10% of fetal bovine serum and 1% of penicillin-streptomycin at 4×10⁵ cells/ml. To each well of a 96 well-microtiter plate, 0.2 ml of the cell suspension was added, and cultured at 37° C. overnight in the presence of 5% carbon dioxide gas. Then, the medium in the wells was replaced with a Dulbecco-modified Eagle medium. To each well, 0.4 μl of a solution of a test substance in water was added and then cultured for 24 hours. The agarobiose obtained in Preparation Example 1 and a commercially available agaro-oligosaccharide (trade name: Agaoligo) were used as test substances. For a negative control, water was added in place of a test substance. After completion of the culture, the medium was removed and the cells were washed with a phosphate buffer. Then, 0.1 ml of a cell-lysis solution (2 mM EDTA (pH 7.8), 1% NP-40) was added and incubated at 37° C. for 10 minutes to obtain an enzyme solution. To 25 it of the enzyme solution, 100 it of a reaction solution (25 mM Tris-HCl (pH 7.4), 0.67% BSA, 0.01% Tween 20, 5 μM FAD, 1 mM G6P, 30 μM NADP, 0.3 mg/ml MTT, 2 U/ml G6PDH (manufactured by Sigma)) was added. At this time, a substrate was added or not added. In the case where a substrate was added, menadion (manufactured by Sigma) was further added at a final concentration of 50 μM into the reaction solution. After incubation at room temperature for 30 minutes, 75 μl of 2N Na₂CO₃ was added to terminate the reaction, and an absorbanc at 590 nm was measured. This activity assay was performed in triplicate. The protein content was measured by using a 50-fold dilution of the enzyme solution with a phosphate buffer and MicroBCA protein assay kit. The test substances were added in such amounts that they reached the concentrations shown in the following table. The QR activity was expressed as a QR activity relative to the control and calculated by the following formula.

QR activity={[(Absorbance in test substance-added section in the presence of substrate)−(Absorbance in test substance-added section in the absence of substrate)]/(Protein content in test substance-added section)}/{[(Absorbance in water-added section in the presence of substrate)−(Absorbance in water-added section in the absence of substrate)]/(Protein content in water-added section)}

Results are shown in Table 3. Table 3 shows the QR activity in cells to which each test substance was added. It was found that the addition of an agaro-oligosaccharide or agarobiose resulted in a significant increase of the QR activity.

TABLE 3

| Test substance | Final concentration | GST activity |
|---|---|---|
| Agaro-oligosaccharide | 25 μg/ml | 1.2 |
| | 50 μg/ml | 2.4 |
| | 100 μg/ml | 2.2 |
| | 200 μg/ml | 2.4 |
| Agarobiose | 25 μM | 1.4 |
| | 50 μM | 1.4 |
| | 100 μM | 1.8 |
| | 200 μM | 2.2 |

### Example 4

### Evaluation of Induction Effect on Expression of Glutathione S-transferase (GST) mRNA

Hepa1c1c7 cells were suspended in a Dulbecco-modified Eagle medium containing 10% of fetal bovine serum and 1% of penicillin-streptomycin at 4×10⁵ cells/ml. To each well of a 6 well-plate, 5 ml of the cell suspension was added, and cultured at 37° C. overnight in the presence of 5% carbon dioxide gas. Then, the medium in the wells was replaced with a Dulbecco-modified Eagle medium. To each well, a solution of a commercially available agaro-oligosaccharide (trade name: Agaoligo) in water as a test substance was added at a final concentration of 100 μg/ml, and then cultured for 16 hours. For a negative control, water was added in place of a test substance. After completion of the culture, the medium was removed and 0.5 ml of RNA iso (manufactured by Takara Bio Inc.) was added. The cells were recovered in a 1.5 ml Eppendorf micro-tube, and left at room temperature for 5 minutes. Thereto 0.1 ml of chloroform was added. The mixture was shaken well until it became milky white. The mixture was left at room temperature for 5 minutes, and centrifuged at 10,000 rpm at 4° C. for 15 minutes. A supernatant was transferred into another Eppendorf micro-tube. Thereto was added 0.25 ml of isopropanol, and mixed well. The mixture was left at room temperature for 10 minutes, and centrifuged at 10,000 rpm at 4° C. for 10 minutes to obtain a precipitate. The precipitate was washed with 0.5 ml of 75% EtOH, centrifuged at 10,000 rpm at 4° C. for 5 minutes, and then dried. The precipitate was dissolved in 20 al of water for injection to obtain a solution of a total RNA in water. A reverse transcription reaction and real time PCR were carried out by using ExScript RT-PCR Kit (manufactured by Takara Bio Inc.). For real time PCR, a primer specific for GST, and a primer specific for transferrin receptor (Tfrc) as a control were used. Measurement was performed by using Smart Cycler II System (manufactured by Cepheid Co.). This activity assay was performed in duplicate. The expression amount of GST mRNA was expressed as a GST mRNA amount relative to the control and calculated by the following formula.

Expression amount of GST mRNA=[(Expression amount of GST mRNA in test substance-added section)/(Expression amount of Tfrc mRNA in test substance-added section)]/[(Expression amount of GST mRNA of water-added section)/(Expression amount of Tfrc mRNA in water-added section)]

Results are shown in Table 4. Table 4 shows the GST mRNA expression amount in cells to which an agaro-oligosaccharide was added. It was found that an agaro-oligosaccharide had a significant GST mRNA expression-inducing activity.

TABLE 4

| Test substance | Expression amount (−fold) |
|---|---|
| 100 μg/ml agaro-oligosaccharide | 4.8 |

### Example 5

### Evaluation of Induction Effect on Expression of Quinone Reductase (QR) mRNA

A QR mRNA expression-inducing activity of an agaro-oligosaccharide was measured in accordance with the method described in Example 4. A commercially available agaro-oligosaccharide (trade name: Agaoligo) was used as a test substance. This activity assay was performed in duplicate. The expression amount of QR mRNA was expressed as a QR mRNA amount relative to the control and calculated by the following formula.

Expression amount of QR mRNA=[(Expression amount of QR mRNA in test substance-added section)/(Expression amount of Tfrc mRNA in test substance-added section)]/[(Expression amount of QR mRNA of water-added section)/(Expression amount of Tfrc mRNA in water-added section)]

Results are shown in Table 5. Table 5 shows the QR mRNA expression amount in cells to which an agaro-oligosaccharide was added. It was found that an agaro-oligosaccharide had a significant QR mRNA expression-inducing activity.

TABLE 5

| Test substance | Expression amount (−fold) |
|---|---|
| 100 μg/ml agaro-oligosaccharide | 4.4 |

### Example 6

### Evaluation of Enhancing Effect on UDP-glucuronosyltransferase (UGT) Activity

A UGT activity was measured by a partially modified method from a method described in B. Burchell, P. Weatherill et al., Methods in Enzymology 77, p 169 (1981). Hepa1c1c7 cells were suspended in a Dulbecco-modified Eagle medium containing 10% of fetal bovine serum and 1% of penicillin-streptomycin at 4×10⁵ cells/ml. To each well of a 12 well-plate, 2 ml of the cell suspension was added, and cultured at 37° C. overnight in the presence of 5% carbon dioxide gas. Then, the medium in the wells was replaced with a Dulbecco-modified Eagle medium. To each well, 4 μl of a solution of a commercially available agaro-oligosaccharide (trade name: Agaoligo) in water as a test substance was added, and then cultured for 24 hours. For a negative control, water was added in place of a test substance. After completion of the culture, the medium was removed and the cells were washed with a phosphate buffer. After the cells were frozen and thawed, 0.2 ml of a reaction solution (0.1M Tris-HCl (pH 7.4), 1 mM MgCl₂, 0.02% Triton X-100, 0.15 mM p-nitrophenol (PNP, manufactured by Nacalai Tesque, Inc.)) was added thereto, stirred well, and incubated in ice for 30 minutes to obtain an enzyme solution. Into a 96 well-mictotiter plate, 80 μl of the enzyme solution was transferred. To wells, 20 al of 20 mM glucuronic acid (manufactured by Wako Pure Chemical Industries, Ltd.) was added or not added. The plate was incubated at 37° C. for 1 hour.

Further, a calibration curve was made using PNP with known concentrations. Then, 100 μl of a 2M glycine buffer (pH 10.4) was added and an absorbance at 405 nm was measured. This activity assay was performed in triplicate. The test substance was added in such an amount that it had the concentration shown in the following table. The UGT activity was expressed as a conjugated PNP amount relative to the control and calculated by the following formula. UGT activity=[(PNP amount in test substance-added section without glucuronic acid)-(PNP amount in test substance-added section with glucuronic acid)]/[(PNP amount in water-added section without glucuronic acid)-(PNP amount in water-added section with glucuronic acid)]

Results are shown in Table 6. Table 6 shows the UGT activity in cells to which an agaro-oligosaccharide was added. It was found that the addition of an agaro-oligosaccharide resulted in a significant increase of the UGT activity.

TABLE 6

| | | UGT activity |
|---|---|---|
| Test substance | Final concentration | (−fold) |
| Agaro-oligosaccharide | 50 μg/ml | 1.1 |
| | 100 μg/ml | 1.4 |
| | 200 μg/ml | 1.4 |

### Example 7

### Evaluation of Induction Effect on Expression of UDP-glucuronosyltransferase (UGT) mRNA

A UGT mRNA expression-inducing activity of an agaro-oligosaccharide was measured in accordance with the method described in Example 4. A commercially available agaro-oligosaccharide (trade name: Agaoligo) was used as a test substance. This activity assay was performed in duplicate. The expression amount of UGT mRNA was expressed as a UGT mRNA amount relative to the control and calculated by the following formula.

Expression amount of UGT mRNA=[(Expression amount of UGT mRNA in test substance-added section)/(Expression amount of Tfrc mRNA in test substance-added section)]/[(Expression amount of UGT mRNA of water-added section)/(Expression amount of Tfrc mRNA in water-added section)]

Results are shown in Table 7. Table 7 shows the UGT mRNA expression amount in cells to which an agaro-oligosaccharide was added. It was found that an agaro-oligosaccharide had a significant UGT mRNA expression-inducing activity.

TABLE 7

| Test substance | Expression amount (−fold) |
|---|---|
| 100 μg/ml agaro-oligosaccharide | 2.0 |

### Example 8

### Evaluation of Enhancing Effect on Intracellular Glutathione (GSH) Content

A GST content was measured by a partially modified method from a method described in Clarissa Gerhauser et al., Cancer Research 57, 272-278 (1997). Hepa1c1c7 cells were suspended in a Dulbecco-modified Eagle medium containing 10% of fetal bovine serum and 1% of penicillin-streptomycin at 4×10⁵ cells/ml. To each well of a 96 well-microtiter plate, 0.2 ml of the cell suspension was added, and cultured at 37° C. overnight in the presence of 5% carbon dioxide gas. Then, the medium in the wells was replaced with a Dulbecco-modified Eagle medium. To each well, 0.4 μl of a solution of a test substance in water was added and then cultured for 24 hours. The agarobiose obtained in Preparation Example 1 and a commercially available agaro-oligosaccharide (trade name: Agaoligo) were used as test substances. For a negative control, water was added in place of a test substance. After completion of the culture, the medium was removed and the cells were washed with a phosphate buffer. The solution was removed, and freeze-thaw of the cells was repeated three times. Thereto was added 0.1 ml of buffer A (125 μM sodium phosphate buffer (pH 7.5), 6.3 mM EDTA) to obtain a cell lysate. To 25 it of the cell lysate, 100 al of a reaction solution (25 mM Tris-HCl (pH 7.4), 1 mM G6P, 30 μM NADP, 2 U/ml G6PDH, 0.25 U/ml glutathione reductase (manufactured by Sigma), 0.6 mM DTNB) was added. After incubation at room temperature for 5 minutes, an absorbance at 405 nm was measured. This activity assay was performed in triplicate. At the same time, as a standard, serial 2-fold dilutions of 2 to 200 al of GST were used in place of the cell lysate. The protein content was measured by using a 50-fold dilution of the cell lysate with a phosphate buffer and MicroBCA protein assay kit. The test substances were added in such amounts that they had the concentrations shown in the following table. The GSH content was expressed as a GSH content relative to the control and calculated by the following formula.

GSH content=[(GSH content in test substance-added section)/(Protein content in test substance-added section)]/[(GSH content in water-added section)/(Protein content in water-added section)]

Results are shown in Table 8. Table 8 shows the GSH content in cells to which each test substance was added.

It was found that the addition of an agaro-oligosaccharide or agarobiose resulted in a significant increase of the GSH content.

TABLE 8

| | | GSH content |
|---|---|---|
| Test substance | Final concentration | (−fold) |
| Agaro-oligosaccharide | 25 μg/ml | 1.4 |
| | 50 μg/ml | 1.6 |
| | 100 μg/ml | 1.5 |
| | 200 μg/ml | 2.4 |
| Agarobiose | 100 μM | 1.3 |
| | 200 μM | 1.9 |

### Comparative Example

### Evaluation of Enhancing Effect of Neoagaro-oligosaccharide on Glutathione S-transferase (GST) Activity and Quinone Reductase (QR) Activity

Enhancing effects on a GST activity and a QR activity were evaluated in the same methods as in Example 1 and Example 3 respectively, using neoagarohexaose as a neoagaro-oligosaccharide. Results are shown in Table 9. Table 9 shows the GST activity and the QR activity in cells to which neoagarohexaose was added. It was found that the addition of neoagarohexaose did not result in a significant increase of the GST activity and the QR activity.

TABLE 9

| | Final | GST activity | QR activity |
|---|---|---|---|
| Test substance | concentration | (−fold) | (−fold) |
| Neoogarohexaose | 100 μM | 1.1 | 1.0 |
| | 200 μM | 1.1 | 1.1 |

### INDUSTRIAL APPLICABILITY

According to the present invention, there are provided an enhancer of a phase II detoxifying enzyme activity or an intracellular glutathione content, which contains at least one compound selected from the group consisting of agar, agarose, a low molecular compound of agarose having 3,6-anhydrogalactopyranose at the reducing end, a compound represented by the above-described formula (Chemical Formula 1), a derivative thereof and a salt thereof, as an active ingredient, and a drug, a food or a feed containing the enhancer. The drug, food or feed containing the enhancer promotes a detoxifying process by the effect of enhancing a phase II detoxifying enzyme activity or an intracellular glutathione content, and therefore, is extremely useful as a drug, a food or a beverage for treatment or prevention of various diseases, in particular, as a drug or a functional food for disease prevention which decreases disease risks.

1-6. (canceled)

7. A method for enhancing a activity of a phase II detoxifying enzyme or a content of intracellular glutathione, comprising a step of administrating a composition containing at least one compound selected from the group consisting of agar, agarose, a low molecular compound of agarose having 3, 6 anhydrogalactopyranose at the reducing end, a compound represented by the following formula (Chemical Formula 1): 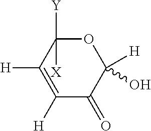
wherein X and Y are H or CH₂OH, provided that Y is H when X is CH₂OH, and Y is CH₂OH when X is H; a derivative thereof, and a salt thereof, as an active ingredient.

8. The method according to claim 7, wherein the low molecular compound of agarose having 3,6-anhydrogalactopyranose at the reducing end is an agaro-oligosaccharide.

9. The method according to claim 8, wherein the agaro-oligosaccharide is a mixture comprising agarobiose, agarotetraose, agarohexaose and agarooctaose.

10. The method according to claim 7, wherein the phase II detoxifying enzyme is glutathione S-transferase, quinone reductase, or UDP-glucuronosyltransferase.

11. The method according to claim 7, wherein the method is for decreasing risks of various diseases caused by toxic substances.

12. The method according to claim 7, wherein the method is for promoting a hepatic function for metabolism of toxic substances.

13. The method according to claim 7, wherein the method is for treating or preventing at least one disease selected from the group consisting of liver inflammation, liver cirrhosis, liver cancer, fatty liver, alcoholic liver disease, cancer, arteriosclerosis, Alzheimer's disease, obesity, and dermatosis.

14. The method according to claim 7, wherein the method is for preventing aging phenomena.

15. The method according to claim 7, wherein the method is for preventing hangover.

16. The method according to claim 15, wherein the step of administrating is administration before or after alcohol intake.

17. A composition comprising:
(A) a compound at least one compound selected from the group consisting of agar, agarose, a low molecular compound of agarose having 3,6-anhydrogalactopyranose at the reducing end, a compound represented by the following formula (Chemical Formula 1): 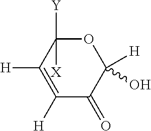
wherein X and Y are H or CH₂OH, provided that Y is H when X is CH₂OH, and Y is CH₂0H when X is H; a derivative thereof, and a salt thereof; and (B) an isothiocyanate and/or a curcumin.
